(19) United States
(12) Patent Application Publication
(10) Pub. No.: US 2026/0340912 A1
(43) Pub. Date: Oct. 29, 2026

(54) METHOD AND X-RAY IMAGING SYSTEM FOR OBTAINING THREE-DIMENSIONAL X-RAY IMAGES OF A SAMPLE

(71) Applicant: Carl Zeiss SBE, LLC, Thornwood, NY (US)

(72) Inventor: Johannes RUOFF, Cupertino, CA (US)

(21) Appl. No.: 19/191,880

(22) Filed: Apr. 28, 2025

(51) Int. Cl. G06T 12/00; A61B 6/02; G06T 15/00

(52) U.S. Cl. CPC G06T 12/00; A61B 6/027; G06T 15/00

(57) ABSTRACT

A method for obtaining three-dimensional x-ray images of a sample, comprises: a) arranging a sample in an object plane of an x-ray imaging system, the sample having a first size in the object plane; b) obtaining two-dimensional x-ray images of a region of interest of the sample including information for different projection angles, the region of interest having a second size in the object plane of at least 0.01 times the first size; and c) reconstructing, computer-implemented, a three-dimensional x-ray image of the region of interest based on the obtained two-dimensional x-ray images.

### FIELD

The present disclosure relates to a method for obtaining three-dimensional x-ray images of a sample and an x-ray imaging system for obtaining three-dimensional x-ray images of a sample.

### BACKGROUND

X-rays are widely used in microscopy because of their short wavelengths and ability to penetrate objects. Three-dimensional (3D) x-ray imaging techniques can be useful to image internal structures of objects. Typically, based on a dataset including x-ray transmission images of a region of interest of a sample that are collected over a large angular range, 3D images of the region of interest are reconstructed. An x-ray imaging system usually comprises a rotatable sample mount to support a sample, an x-ray source configured to illuminate the region of interest of the sample, and a position-sensitive x-ray detector configured to record x-rays transmitted through the region of interest of the sample.

### SUMMARY

The present disclosure seeks to provide improve x-ray imaging of a sample with an x-ray imaging system.

According to a first aspect, a method for obtaining three-dimensional x-ray images of a sample is provided. The method comprises the steps:
a) arranging a sample in an object plane of an x-ray imaging system, the sample having a first size in the object plane;
b) obtaining two-dimensional x-ray images of a region of interest of the sample including information for different projection angles, the region of interest having a second size in the object plane of at least 0.01 times the first size; and
c) reconstructing, computer-implemented, a three-dimensional x-ray image of the region of interest based on the obtained two-dimensional x-ray images.

With the proposed method, it can be possible to analyze a significant portion of a relatively large sample by three-dimensional x-ray imaging.

The method can be applied for x-ray imaging of a relatively large region of interest of a large sample. The sample is, for example, a flat extended object. The sample is, for example, a wafer. The wafer includes, for example, electronic and/or semiconductor components. Just as an example, the x-ray imaging system may be used to inspect the wafer to investigate the quality of packaging of electronic components of the wafer. For example, the quality of mechanical and electrical bonding (e.g., buried interconnections) of the electronic components may be controlled. However, the sample may also be another object than a wafer. The sample can be, for example, a printed circuit board, a battery, a panel or the like.

The sample has, for example, a rectangular shape, a squared shape and/or a circular shape in its main plane of extension. When the sample is arranged in the object plane of the x-ray imaging system, the main plane of extension of the sample is, for example, arranged parallel to the object plane.

The size of the sample in the object plane (first size) is, for example, the size of the sample in its main plane of extension.

The size of the sample in the object plane (first size) is, for example, at least 1,000 square millimeters (mm²), at least 3,000 mm² (e.g., at least 5,000 mm², at least 10,000 mm², at least 30,000 mm², at least 50,000 mm², at least 70,000 mm² and/or at least 360,000 mm²).

The size of the region of interest in the object plane (second size) is, for example, at least 0.01 times (e.g., at least 0.1 times, at least 0.3 times, at least 0.5 times, at least 0.7 times and/or at least 0.9 times) the size of the sample (first size). The two-dimensional x-ray images, for example, at least 1% (e.g., at least 10%, at least 30%, at least 50%, at least 70% and/or at least 90%) of the entire sample are obtained in step b). A three-dimensional x-ray image of the fraction of the sample can be reconstructed in step c).

The region of interest may include a connected region (e.g., a single connected region) and/or may include an unconnected region. The connected region includes, for example, a simply-connected region and/or a non-simply connected region. The unconnected region includes, for example, two or more disjoint sub-regions.

The second size of the region of interest in the object plane may also be the same as the first size of the sample. The two-dimensional x-ray images of the entire sample may be obtained in step b). A three-dimensional x-ray image of the entire sample may be reconstructed in step c).

The first size, the second size and a third size (see below) are each, for example, the size of an area (i.e. a size in two dimensions).

In step b), x-ray images covering together the entire region of interest are obtained. The individual images may cover only a fraction of the region of interest.

The method is, for example, a method for transmission x-ray imaging. Transmission x-ray imaging means that the x-rays impacting on the region of interest of the sample are partly transmit by the region of interest and are partly absorbed by the region of interest. The position-dependent transmitted portion of the x-rays can be detected by an x-ray detector assembly (e.g., a position-sensitive x-ray detector assembly) as a two-dimensional image.

The method can be used for three-dimensional imaging of the region of interest of the sample. For example, two-dimensional transmission x-ray images of the region of interest are obtained, wherein each image includes information for different projection angles. Based on the two-dimensional transmission images, a three-dimensional image of the region of interest can be reconstructed to reveal interior structures of the region of interest. The three-dimensional image is, for example, reconstructed computer-implemented. An x-ray imaging system used in the method comprises, for example, a control device for reconstructing the three-dimensional image(s). The x-ray imaging system is, for example, an x-ray three-dimensional imaging system obtaining three-dimensional images by x-ray laminography.

The object plane of the x-ray imaging system is, for example, defined by a support surface of the system for supporting the sample.

An x-ray imaging system comprises, for example, a sample mount assembly. The sample mount assembly comprises, for example, a (e.g., non-moving) base and a sample mount (moving stage) attached movably to the base. The sample mount comprises, for example, the support surface for supporting the sample. The support surface is, for example, a flat surface (e.g., flat on a micrometer and/or nanometer scale).

The movable sample mount (“xy-stage”) can be movable laterally in a plane parallel to the support surface and/or the object plane.

The sample mount and/or support surface of the sample mount has, for example, an opening (e.g., through opening). The opening is, for example, configured for transmission of x-rays from an x-ray source towards the sample or from the sample towards a detector assembly. The opening may instead or in addition also be configured for accommodating a protruding portion of the x-ray source at least partially. Instead of an opening for transmission of x-rays, the sample mount and/or the support surface may also have an x-ray transmissive window for transmission of x-rays towards the sample or from the sample.

An element being x-ray transmissive means herein, for example, that it has an x-ray transmission such that more than 50% of the x-rays generated by at least one x-ray target of the x-ray source and having energies greater than one-half of the selected maximum focused electron energy are transmitted through the respective element.

In step a), the sample is, for example, arranged on the support surface of the sample mount. The sample is, for example, arranged manually and/or by a robot-like unit on the support surface. Further, the sample is, for example, arranged on the support surface of the sample mount such that a region of interest of the sample is arranged on the opening of the x-ray transmissive portion of the sample mount.

Obtaining two-dimensional x-ray images of the region of interest of the sample including information for different projection angles generally includes irradiating the region of interest with x-rays from an x-ray source. Furthermore, x-rays transmitted through the region of interest are typically detected by an x-ray detector assembly. The field-of-view of the detector assembly is, for example, relatively large such that for each two-dimensional image, the region of interest or a portion of the region of interest is irradiated with x-rays having different angles of incident. Further, scanning the entire region of interest is, for example, achieved by laterally moving the sample in a plane parallel to the object plane. The sample is, for example, moved laterally with respect to the (e.g., fixed) x-ray source and the (e.g., fixed) x-ray detector assembly.

According to an embodiment, the two-dimensional x-ray images of the region of interest are obtained by an x-ray detector assembly having a field-of-view with a third size. In addition, the second size of the region of interest can be larger than the third size and/or the second size of the region of interest is larger than the third size by a factor of 10 or more (e.g., 100 or more, 1,000 or more and/or 10,000 or more).

That area of the sample which is scanned with the method (i.e., the region of interest) can be larger (e.g., many times larger) than the field-of-view of the detector assembly.

The field-of-view of the x-ray detector assembly depends, for example, on a vertical position of the sample within the x-ray cone of the x-ray source. The vertical position is a position with respect to a vertical direction of the x-ray imaging system, the vertical direction being oriented perpendicular to the object plane of the system.

The field-of-view of the x-ray detector assembly is, for example, defined at a surface of the sample, the surface facing the x-ray detector assembly.

According to an embodiment, the two-dimensional x-ray images of the region of interest used to reconstruct the three-dimensional x-ray image of the region of interest are obtained within a time span of four hours or less (e.g., three hours or less, two hours or less, one hour or less and/or half an hour or less).

By scanning the relatively large region of interest within a moderate time span, a satisfying throughput rate of samples is possible.

For example, it is possible for a geometric magnification of the x-ray imaging for obtaining the two-dimensional x-ray images of the region of interest to be only moderate in order to image a relatively large region of interest within a relatively short time span. X-ray imaging is, for example, performed with a geometric magnification of 30 or less (e.g., 20 or less, 10 or less and/or 5 or less).

The geometric magnification of an x-ray imaging system is defined by the geometric arrangement of an x-ray source, a sample and a detector. The geometric magnification of the x-ray imaging system is, for example, equal to a ratio of a first distance and a second distance. The first distance is a distance between the x-ray source and the x-ray detector assembly. The second distance is a distance between the x-ray source and the region of interest of the sample.

For imaging with only moderate geometric magnification, the first distance between the x-ray source and the x-ray detector assembly should be relatively small (e.g., 10 millimeters (mm) or less and/or 5 mm or less).

According to an embodiment, the two-dimensional x-ray images of the region of interest are obtained by use of an x-ray source irradiating the region of interest with x-rays and an x-ray detector assembly detecting x-rays transmitted through the region of interest. Furthermore, the sample is moved laterally in step b) within the image plane and relative to the x-ray source and the x-ray detector assembly such that each portion of the region of interest is irradiated by x-rays of an x-ray cone of the x-ray source under different angles of incident for the different lateral positions to obtain the two-dimensional x-ray images of the region of interest including the information for the different projection angles.

The x-ray detector assembly comprises a detector unit which is arranged, for example, parallel to the object plane. Thus, the detector unit of the x-ray detector assembly can be arranged parallel to the main plane of extension and/or parallel to the surface of the sample. Having the x-ray detector assembly with the detector unit arranged parallel to the sample—instead of at an oblique angle—helps allow for the x-ray detector assembly to be arranged very close to the sample without the risk of colliding with the sample.

The sample can be moved in a horizontal plane between the x-ray source and the x-ray detector assembly—instead of rotating the sample around a vertical axis. The sample is, for example, moved laterally such that the region of interest of the sample is moved through the x-ray cone of the x-ray source.

The sample is, for example, moved only laterally. In other words, the sample is, for example, not rotated relative to the x-ray source and/or the x-ray detector assembly.

According to an embodiment, the sample is moved laterally in a pattern including a meander pattern, a spiral pattern and/or a pattern of concentric circles.

By using the movable sample amount (“xy-stage”), any position of the sample with respect to its surface can be imaged by the x-ray imaging system.

A meander pattern includes, for example, a path including several straight path portions. A spiral pattern and a pattern of concentric circles each includes, for example, curved path portions. Each position of the respective pattern is approached by setting the lateral coordinates of the sample mount (“xy-stage”).

According to an embodiment, the two-dimensional images of the region of interest are obtained by a step-and-shoot process. The step-and-shoot process includes for each of multiple predetermined lateral positions of the sample:
laterally moving the sample to a predetermined lateral position without obtaining x-ray images of the region of interest during a first time span, and
irradiating the region of interest in the predetermined lateral position of the sample with x-rays without moving the sample during a second time span.

By using the step-and-shoot approach, the sample (i.e. sample mount with the sample) can be moved during the first time span quickly from one position to the next position (“step”) and then comes to a complete stop. Further, the sample can be moved during the first time span without taking x-ray images of the sample. For example, the x-ray source and/or the x-ray detector assembly is/are, for example, shut off during the first time span. Then, during the second time span the sample remains stationary and an x-ray exposure of the sample can be carried out (“shoot”). Hence the x-ray source and/or the x-ray detector assembly can be switched on again during the second time span.

By using the step-and-shoot process for obtaining the x-ray images, it is possible to avoide a blur of the x-ray images due to motion of the sample during the x-ray exposure. For example, it can be avoided that the image from a specific feature moves across a few pixels during the exposure.

The first time span is part of an overhead time of the x-ray imaging process. Further, the second time span is the exposure time. A ratio of the first time span and the second time span defines an x-ray imaging efficiency.

The first time span is, for example, a factor of 0.2 or less (e.g., 0.1 or less) of the second time span. With a ratio of 20% or less (e.g., 10% or less) of the overhead time to the exposure time an efficient x-ray imaging process can be carried out.

For example, in the step-and-shoot approach it is desirable to minimize the stepping time (i.e. the first time span) in order to maximize the exposure time (i.e. the second time span). In addition, generally, the shorter the stepping time, the larger the acceleration and deceleration capabilities of the sample mount assembly are. The sample mount assembly is, for example, configured for laterally moving the sample mount with an acceleration/deceleration of 10 meters per second squared (m/s²) (~1 g) or more (e.g., 20 m/s² (~2 g) or more and/or 30 m/s² (~3 g) or more).

According to an embodiment, the two-dimensional x-ray images of the region of interest are obtained by irradiating the region of interest such that angles of incident of the x-rays hitting the region of interest each have a value of 60° or less (e.g., 50° or less and/or 40°or less).

The x-ray detector assembly includes, for example, a scintillator element for converting incoming x-rays into detectable light and a detector unit (e.g., a detector array, a two-dimensional array, CCD, CMOS) for detecting the detectable light.

Optionally, the x-ray detector assembly may include-instead of a scintillator element for converting incoming x-rays into detectable light and a detector unit for detecting the detectable light-a detector unit for directly detecting x-ray light (“direct detector” or “photon counting detector”). In this example, the x-ray detector assembly may be configured for detecting electrons released by the x-rays. In the following, the x-ray detector assembly is mainly described as having a scintillator element for converting incoming x-rays into detectable light and a detector unit for detecting the detectable light. The features described with respect to such an x-ray detector assembly apply in an analog manner to the case of an x-ray detector assembly with a detector unit for directly detecting x-ray light.

The x-ray detector assembly may also include, for example, a (magnifying or non-magnifying) optics unit for transferring the detectable light from the scintillator element to the detector unit. The x-ray detector assembly may include a (magnifying or non-magnifying) fiber optic plate (FOP) for guiding the detectable light from the scintillator element to the detector unit.

The main plane of extension of the scintillator element and/or a main plane of extension of the detector unit (e.g., the plane of extension of a two-dimensional pixel array of the detector unit) are, for example, arranged parallel to each other. In case that a fiber optic plate is used, also a main plane of extension of the fiber optic plate can be arranged parallel to the main plane of extension of the scintillator element and/or the main plane of extension of the detector unit.

The x-ray images of the region of interest are obtained, for example, by irradiating the region of interest such that each portion of the region of interest is irradiated with a respective x-ray beam having a different oblique angle of incident. However, when the region of interest is irradiated with an x-ray beam having an angle of incident deviating from a normal incidence (i.e. deviating from a 90° angle between the impacting x-ray and a main plane of extension of the x-ray detector assembly), a single x-ray beam will cause the scintillator element to admit scintillation photons along the whole beam path of the x-ray beam in the scintillator element which has a finite thickness. Further, the scintillation photons from a single x-ray beam will be detected by more than one pixel of the detection unit causing a loss of spatial resolution of the imaging process. The amount of blurring depends on the thickness of the scintillator element and increases with increasing thickness of the scintillator element is.

In addition, another factor which can partially compensate the loss of resolution for oblique x-rays can be taken into account. Suppose two parallel x-rays spaced apart from each other by the pixel width of the detector unit. Then for normal incidence, these rays can clearly be separated, since each one will be detected by one pixel. However, when tilted the horizontal projection of the separation gets increased, which means that the distance between the x-rays at the entry points of the pixels gets increased. Conversely, this means that pixels with a given size can resolve x-rays with smaller distances.

Choosing the value of angle of incident to be 60° or less (e.g., 50° or less and/or 40° or less), it is possible to reduce the loss in spatial resolution of the obtained x-ray image.

For example, the applicant has found that taken into account both above-described effects (loss of spatial resolution and compensation effect) and assuming exemplarily a ratio of the thickness D of the scintillator element and the pixel size p of D/p=2.5, for an angle of incident of 30° or smaller, an incoming x-ray beam is detected by one pixel of the detector unit. In an embodiment, for an angle of incident of 40°, an incoming x-ray beam is distributed over 1.3 pixels of the detector unit. In an embodiment, for an angle of incident of 50°, an incoming x-ray beam is distributed over 2.3 pixels of the detector unit.

In an embodiment, for an angle of incident of 60°, an incoming x-ray beam is distributed over 3.8 pixels.

Hence, choosing an angle of incident of 40° or smaller, the loss of spatial resolution due to oblique irradiation of the sample can be almost neglectable. By choosing an angle of incident of 50° or smaller, the loss of spatial resolution due to oblique irradiation of the sample can be limited to about a factor of two.

Angles of incident of the x-rays hitting the region of interest having a value of 60° or less (e.g., 50° or less and/or 40° or less) cab be desirable for an x-ray detector assembly comprising a detector unit and a scintillator element each having a flat shape and being arranged parallel to the object plane.

However, in some embodiments the two-dimensional x-ray images of the region of interest are obtained by irradiating the region of interest such that angles of incident of the x-rays hitting the region of interest each can have any value including values exceeding 40°, 50° or even 60° (e.g., values of 80° or less and/or 70° or less). For example, in an embodiment of an x-ray detector assembly comprising a curved and/or semi-spherical shape (e.g., with a detector unit and a scintillator element each having a curved and/or semi-spherical shape) such that the x-ray source faces a concave surface of the x-ray detector assembly, a value of the angles of incident of the x-rays hitting the region of interest may exceed 40°, 50° or even 60° without causing a loss of spatial resolution of the imaging process.

According to an aspect, an x-ray imaging system for obtaining three-dimensional images of a sample is provided. The x-ray imaging system comprises:
a moveable sample stage for supporting the sample in an object plane of the system,
an x-ray source for emitting x-rays towards a region of interest of the sample, and
an x-ray detector assembly for detecting x-rays transmitted through the region of interest of the sample,
wherein the system is configured for
   obtaining two-dimensional x-ray images of the region of interest including information for different projection angles, the sample having a first size in the object plane, and the region of interest having a second size in the object plane of at least 0.01 times the first size of the sample, and
   reconstructing, computer-implemented, a three-dimensional x-ray image of the region of interest based on the obtained two-dimensional x-ray images.

The x-ray source comprises, for example, a vacuum chamber. Further, the x-ray source comprises, for example, a pump for evacuating the vacuum chamber. The x-ray source further comprises, for example, an electron source accommodated in the vacuum chamber. The electron source is configured for emitting an electron beam towards an x-ray target of the x-ray source. The electron source includes, for example, a cathode and an anode and the like for generating electrons and for accelerating the generated electrons.

The x-ray source comprises, for example, one or more electron optics units for directing, deflecting and/or shaping the electron beam emitted from the electron source. The electron optics include, for example, one or more magnetic lenses for focusing the electron beam and/or one or more deflection units for deflecting the electron beam.

The x-ray source further comprises, for example, an x-ray target. The x-ray target is configured for emitting x-rays when bombarded with the focused electron beam. A material of the at least one x-ray target comprises, for example, tungsten (W), copper (Cu), chromium (Cr), molybdenum (Mo), rhodium (Rh) and/or platinum (Pt). The x-rays generated by the at least one x-ray target include, for example, characteristic lines determined by the target's composition and broad bremsstrahlung radiation.

The x-ray source includes, for example, a carrier element carrying the x-ray target (or carrying multiple of the x-ray targets which can be selected by directing the electron beam accordingly). The carrier element is, for example, x-ray transmissive. The carrier element forms, for example, a vacuum window of the vacuum chamber. Optionally, an additional vacuum window may be provided. A material of the carrier element and/or the vacuum window includes, for example, atomic elements having atomic numbers less than 14. The material of the carrier element and/or the vacuum window includes, for example, beryllium (Be), diamond, boron carbide (B₄C), silicon carbide (SiC), aluminum (Al), and/or beryllium oxide (BeO). The material of the carrier element and/or the vacuum window can be diamond.

The carrier element and/or the vacuum window being x-ray transmissive means, for example, that it has an x-ray transmission such that more than 50% of the x-rays generated by the at least one x-ray target having energies greater than one-half of the selected maximum focused electron energy are transmitted through the carrier element.

The carrier element has, for example, a sufficiently high thermal conductivity to provide a thermal conduit to prevent thermal damage (e.g., melting) of the x-ray target. Further, the carrier element can, for example, also provide an electrically conductive path to dissipate electric charge from the at least one x-ray target and/or the carrier element itself.

The x-ray source is, for example, a transmission target type x-ray source. The electron beam can strike the at least one x-ray target of the x-ray source at its backside and the at least one x-ray target can emit x-rays at its front side, the emitted x-rays can be used to irradiate the sample.

The x-ray source can generate diverging x-rays, i.e. a cone (conus) of x-rays.

The x-ray detector assembly is, for example, a position-sensitive x-ray detector assembly. The x-ray detector assembly is, for example, configured for position-sensitive x-ray detection of x-rays transmitted through the region of interest of the sample. The x-ray detector assembly is, for example, configured for converting incoming x-rays into light of longer wavelength, e.g., ultraviolet light, visible light or infrared light. The x-ray detector assembly includes, for example, a scintillator material (scintillator element) at an entrance window of the detector assembly for converting the x-rays into detectable light and a detector unit (e.g., a detector array, a CCD or CMOS array) for detecting the detectable light. The x-ray detector assembly further includes, for example, a transfer unit for transferring the detectable light from the scintillator element to the detector unit. The transfer unit comprises, for example, a (magnifying or non-magnifying) optics unit. The transfer unit may also comprise, for example, a (magnifying or non-magnifying) fiber optic plate.

A fiber optic plate (also called faceplate) comprises, for example, numerous optical fibers, each fiber being configured to guide light from the scintillator element to the detector unit. The fiber optic plate comprises, for example, hundreds, thousands or even millions of coherent optical fibers. The fibers are, for example, fused into a matrix material. For example, each pixel of the two-dimensional array of the detector unit is coupled to one or more fibers of the fiber optic plate. The fiber optic plate comprises, for example, an input face coupled (e.g., directly) to the scintillator element and an output face coupled (e.g., directly) to the detector unit. The fibers of the fiber optic plate optically connect numerous input points at the input face to corresponding output points of the output face. The fiber optic plate provides a one-to-one image transfer from the input face (and hence from the scintillator element) to the output face (and hence to the detector unit).

A fiber optic plate can allow for a much more compact configuration of the x-ray detector assembly compared to an optics unit. The reason is that no lenses or mirrors and no focusing distance are used in the x-ray detector assembly. Hence, using the fiber optic plate, the x-ray detector assembly can be configured with a shorter total length reducing the installation space for the x-ray detector assembly. Image distortions can be much smaller when using a fiber optic plate for light transfer compared to lens-based transfer optics. A fiber optic plate can have a higher light transmission rate than an imaging optic. A fiber optic plate can be configured for absorbing stray light during transmission and, hence, a signal-to-noise ratio (SNR) of the x-ray imaging can be improved.

The detector unit includes, for example, a two-dimensional detector array. The detector unit is, for example, a position-sensitive detector unit. The detector unit is, for example, configured for detecting ultraviolet, visible and/or infrared light.

A pixel size of the detector unit is, for example, 15 micrometers (μm) or less (e.g., 10 μm or less, 5 μm or less, 3 μm or less, 2 μm or less and/or 1 μm or less).

The detector unit may include, for example, a CMOS sensor (Complementary Metal-Oxide Semiconductor sensor). For example, the detector unit includes an active pixel sensor (APS) based on CMOS technology (CMOS-APS) and/or a scientific CMOS sensor. The detector unit may optionally include a CCD sensor (Charged Coupled Device sensor).

The x-ray detector assembly may further comprise, for example, an x-ray transmissive entrance window. Further, the scintillator element is, for example, arranged adjacent and/or attached to the entrance window. The scintillator element is, for example, attached to an inner surface of the entrance window by glueing or another suitable manner.

The scintillator element has, for example, a thickness in a direction perpendicular to its main plane of extension between 1 μm and 500 μm (e.g., between 1 μm and 200 μm, between 1 μm and 50 μm, between 1μm and 30 μm and/or between 1 μm and 20 μm).

By having a small thickness of the scintillator element, an amount of blurring due to the scintillation photons from a single x-ray beam being detected by more than one pixel of the detection unit can be reduced.

A refractive index of the material of the scintillator element is, for example, between 1.50 and 2.20.

According to an embodiment, the x-ray detector assembly has a field-of-view with a third size. In addition, the second size of the region of interest is larger than the third size and/or the second size of the region of interest is larger than the third size by a factor of 10 or more (e.g., 100 or more, 1,000 or more and/or 10,000 or more).

According to an embodiment, the x-ray imaging system comprises a sample stage assembly including the sample stage. The sample stage assembly can be configured for moving the sample laterally, parallel to the image plane and relative to the x-ray source and the x-ray detector assembly.

According to an embodiment, the system is configured to move the sample laterally relative to the x-ray source to different predetermined lateral positions such that each portion of the region of interest is irradiated by x-rays of an x-ray cone of the x-ray source under different angles of incident for the different lateral positions to obtain the two-dimensional x-ray images of the region of interest including the information for the different projection angles.

According to an embodiment of the second aspect, the x-ray detector assembly comprises a detector unit and/or a scintillator element each having a flat shape and being arranged parallel to the object plane.

In a first variant of this embodiment, the detector unit is a detector unit for detecting detectable light emitted by the scintillator element. In this first variant, the x-ray detector assembly comprises the detector unit and the scintillator element each having a flat shape and being arranged parallel to the object plane.

In a second variant of this embodiment, the detector unit is a detector unit for directly detecting x-ray light (i.e. the x-ray detector assembly does not comprise a scintillator element). In this second variant, the x-ray detector assembly comprises the detector unit having a flat shape and being arranged parallel to the object plane.

In the following, the first variant is described in more detail. Features described with respect to the first variant apply—if applicable—mutatis mutandis to the second variant.

For example, a main plane of extension of the flat detector unit and a main plane of extension of the flat scintillator element are each arranged parallel to the object plane.

Thus, both the detector unit and the scintillator element of the x-ray detector assembly are arranged parallel to the main plane of extension and/or to the surface of the sample. Having the x-ray detector assembly with the detector unit and the scintillator element arranged parallel to the sample—instead of at an oblique angle—the x-ray detector assembly can be arranged close to the sample without the risk of colliding with the sample.

Moreover, the sample can be moved in a horizontal plane between the x-ray source and the x-ray detector assembly—instead of rotating the sample around a vertical axis. The sample is, for example moved laterally such that the region of interest of the sample is moved through the x-ray cone of the x-ray source.

Arranging the x-ray detector assembly close to the sample can help allow imaging with a moderate geometric magnification. This means that a relatively large region of interest can be imaged within a moderate time duration.

The geometric magnification of the x-ray imaging system is, for example, equal to a ratio of a first distance and a second distance. The first distance is a distance between the x-ray source and the x-ray detector assembly. Further, the second distance is a distance between the x-ray source and the region of interest of the sample. For example, for imaging with only moderate geometric magnification, the first distance between the x-ray source and the x-ray detector assembly is chosen relatively small (e.g., 10 mm or less and/or 5 mm or less).

Having the detector assembly, for example the detector unit and the scintillator element of the detector assembly, configured flat and arranged parallel to the object plane (that means parallel to the main plane of extension of the sample) provides the same geometric magnification for each pixel of the detector unit. A blurring effect can result because of an increasing number of pixels of the detector unit detecting a single x-ray beam. In other words, a single x-ray beam can be smeared out over an increasing number of pixels of the detector unit. This blurring effect can occur for every exemplary portion within the region of interest, whenever this portion is projected to an edge of the detector unit of the x-ray detector assembly.

According to an embodiment of the second aspect, the x-ray detector assembly comprises a detector unit and/or a scintillator element each having a curved and/or semi-spherical shape such that the x-ray source faces a concave surface of the detector unit and/or of the scintillator element.

In a first variant of this embodiment, the detector unit is a detector unit for detecting detectable light emitted by the scintillator element. In this first variant, the x-ray detector assembly can comprise the detector unit and the scintillator element each having a curved and/or semi-spherical shape such that the x-ray source faces a concave surface of the detector unit and of the scintillator element.

In a second variant of this embodiment, the detector unit is a detector unit for directly detecting x-ray light (i.e. the x-ray detector assembly does not comprise a scintillator element). In this second variant, the x-ray detector assembly can comprise the detector unit having a curved and/or semi-spherical shape such that the x-ray source faces a concave surface of the detector unit.

In the following, the first variant is described in more detail. Features described with respect to the first variant apply—if applicable—mutatis mutandis to the second variant.

The scintillator element and the detector unit are, for example, arranged parallel to each other (in the sense of parallel curves).

The detector unit and the scintillator element have, for example, each a semi-spherical shape with a source region of the x-ray source arranged in the center of a sphere corresponding to the semi-spherical shape. In this case, the scintillator element and the detector unit can be arranged concentrically to each other.

Having a curved and/or semi-spherical shape of the detector unit and the scintillator element, the x-rays emitted from the source region of the x-ray source and transmitted through the region of interest of the sample can impact a surface of the scintillator element at normal incidence. Hence, it is possible to avoid a loss of spatial resolution due to blurring caused by an oblique passing of the x-rays through the scintillator element.

The geometric magnification may no longer be constant across the field-of-view of the detector assembly, because (although the first distance between the x-ray source and the x-ray detector assembly is constant for each detector pixel) the second distance between the x-ray source and the sample is not constant for each detector pixel due to the flatness of the sample. Because the geometric magnification is given by the ratio of the first distance to the second distance, the geometric magnification can also vary across the field-of-view of the x-ray detector assembly.

A semi-spherical shape is, for example, a portion of a spherical shape (e.g., a hemispherical shape).

According to an embodiment, the x-ray detector assembly comprises:
multiple segments, each segment including a flat detector unit and/or a flat scintillator element arranged in a plane parallel to the object plane,
multiple segments, each segment having a curved and/or semi-spherical shape,
multiple Fresnel-like segments, and/or
multiple segments arranged tilted to each other to approximate a curved and/or semi-spherical shape.

In a first variant of this embodiment, each detector unit is a detector unit for detecting detectable light emitted by the corresponding scintillator element. In this first variant, the x-ray detector assembly can comprise multiple segments, each segment including a flat detector unit and a flat scintillator element arranged in a plane parallel to the object plane.

In a second variant of this embodiment, each detector unit is a detector unit for directly detecting x-ray light (i.e. the x-ray detector assembly does not comprise a scintillator element). In this second variant, the x-ray detector assembly can comprise multiple segments, each segment including a flat detector unit arranged in a plane parallel to the object plane.

In the following, the first variant is described in more detail. Features described with respect to the first variant apply—if applicable—mutatis mutandis to the second variant.

Having a detector assembly comprising multiple segments, wherein each segment is flat and arranged in a plane parallel to the object plane, can help provide for a possibility to increase the total sensitive pixel area of the x-ray detector assembly. Thus, a field-of-view of the x-ray detector assembly can be increased.

The segments can be arranged very close to each other to minimize gaps between the segments. Further, remaining gaps between the segments can be taken into account in the algorithms for reconstruction of the three-dimensional image of the region of interest.

Having a detector assembly comprising multiple segments, wherein each segment has a curved and/or semi-spherical shape, can help provide at least some of the above-described features of curved and/or semi-spherical detector shapes.

At the same time the individual segments may have different thicknesses (Fresnel-like segments) and/or different distances from the source region of the x-ray source in order to compensate for the above-described non-homogeneous magnification of a single curved and/or semi-spherical shape. In other words, the individual segments may have different thicknesses and/or different distances from the source region of the x-ray source such that a distance difference between the x-ray source and the sample for the different detector pixels is compensated. The segments can be formed as concentric semi-spherical elements. The innermost segment is, for example, a spherical section (spherical cap) with a circular base. The further segments surround, for example, the innermost segment and have each an annular shape.

According to an aspect, a computer program is provided. The computer program comprises instructions which, when the program is executed by a computer, cause the computer to carry out at least some steps of the above-described method.

A computer program (computer program product) may be embodied as a memory card, USB stick, CD-ROM, DVD or as a file which may be downloaded from a server in a network. For example, such a file may be provided by transferring the file comprising the computer program product from a wireless communication network.

According to an aspect, a control device for an x-ray imaging system is provided. The control device is configured to perform at least some steps of the above-described method.

The embodiments and features described with reference to the first aspect of the present disclosure apply mutatis mutandis to the second to fourth aspects of the present disclosure and vice versa.

Further possible implementations or alternative solutions of the disclosure also encompass combinations-that are not explicitly mentioned herein-of features described above or below with regard to the embodiments. The person skilled in the art may also add individual or isolated aspects and features to the most basic form of the disclosure.

### BRIEF DESCRIPTION OF THE DRAWINGS

Further embodiments, features and aspects of the present disclosure will become apparent from the subsequent description and dependent claims, taken in conjunction with the accompanying drawings, in which:

FIG. 1 shows a schematic cross-section view of an x-ray imaging system for imaging a sample according to an embodiment;

FIG. 2 shows a view similar as FIG. 1 but with the sample moved laterally according to an embodiment;

FIG. 3 shows a view similar as FIG. 2 but with the sample moved further laterally according to an embodiment;

FIG. 4 shows a view similar as FIG. 3 but with the sample moved further laterally according to an embodiment;

FIG. 5 shows a view similar as FIG. 4 but with the sample moved further laterally according to an embodiment;

FIG. 6 illustrates a pattern for moving the sample of FIG. 1 according to an embodiment;

FIG. 7 illustrates a pattern for moving the sample of FIG. 1 according to an embodiment;

FIG. 8 illustrates a pattern for moving the sample of FIG. 1 according to an embodiment;

FIG. 9 illustrates a step-and-shoot process for moving the sample of FIG. 1 according to an embodiment;

FIG. 10 shows a schematic cross-section view of an x-ray imaging system for imaging a sample according to an embodiment;

FIG. 11A shows a schematic cross-section view of an x-ray imaging system for imaging a sample according to an embodiment;

FIG. 11B shows a top view of detector segments of an x-ray detector assembly of the x-ray imaging system of FIG. 11A according to an embodiment;

FIG. 11C shows a variant of the x-ray imaging system of FIG. 11A according to an embodiment;

FIG. 12A shows a schematic cross-section view of an x-ray imaging system for imaging a sample according to a further embodiment;

FIG. 12B shows a top view of detector segments of an x-ray detector assembly of the x-ray imaging system of FIG. 12A according to an embodiment;

FIG. 13 shows a schematic cross-section view of an x-ray imaging system for imaging a sample according to an embodiment;

FIG. 14 shows a flowchart illustrating a method for obtaining three-dimensional x-ray images of a sample according to an embodiment; and

FIG. 15 shows a control device of the x-ray imaging system of FIG. 1 according to an embodiment.

### DETAILED DESCRIPTION

In the Figures, like reference numerals designate like or functionally equivalent elements, unless otherwise indicated.

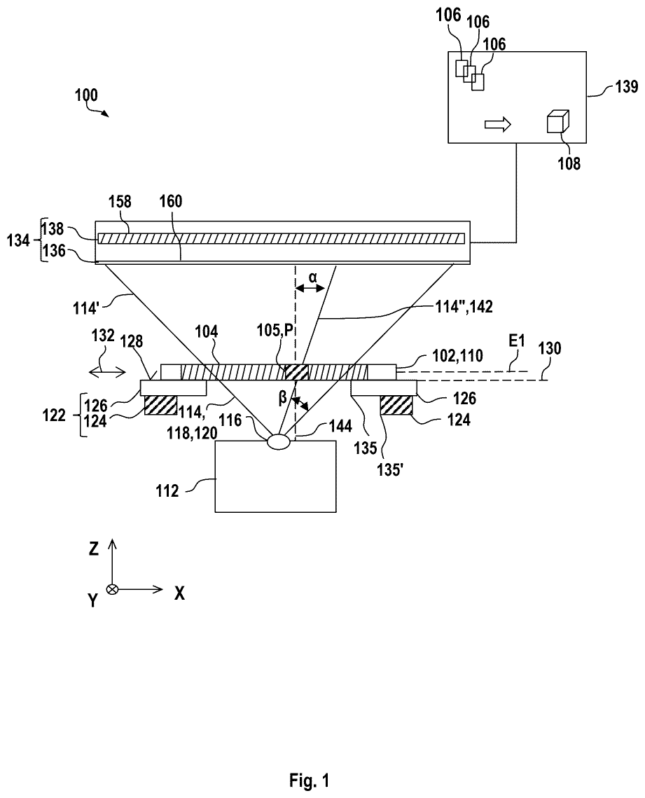

FIG. 1 shows a schematic view of an x-ray imaging system 100 according to an embodiment. The x-ray imaging system 100 is used for imaging a sample 102, for example a region of interest 104 of the sample 102. The x-ray imaging system 100 is configured to obtain two-dimensional transmission images 106 of the region of interest 104 including information for different projection angles α. Based on the two-dimensional transmission images 106, a three-dimensional (3D) image 108 of the region of interest 104 is reconstructed to reveal interior structures of the region of interest 104.

The sample 102 is, for example, a flat object extended in a main plane E1 (e.g., the xy-plane in FIG. 1). The sample 102 is, for example, a wafer 110 comprising electronic and/or semiconductor components. Just as an example, the x-ray imaging system 100 may be used to inspect the wafer 110 to investigate the quality of packaging of electronic components of the wafer 110. For example, the quality of mechanical and electrical bonding (e.g., buried interconnections) of the electronic components may be controlled.

The x-ray imaging system 100 comprises an x-ray source 112 for emitting x-rays 114. The x-rays 114 are emitted from a source region 116 of the x-ray source 112. The x-ray source 112 emits a diverging beam 118 of x-rays 114. In other words, the x-ray source 112 emits a cone 120 of x-rays 114. The sample 102 is arranged within the x-ray emission cone 120.

The x-ray imaging system 100 further comprises a sample mount assembly 122 for supporting the sample 102. The sample mount assembly 122 comprises a (e.g., non-moving) base 124 and a moveable sample mount 126 (moving stage) attached movably to the base 124. The sample mount 126 comprises, for example, a support surface 128 for supporting the sample 102. The support surface 128 defines, for example, an object plane 130 of the x-ray imaging system 100.

The sample mount 126 can be moved laterally (arrow 132 in FIG. 1) with respect to the base 124 of the sample mount assembly 122. The sample mount 126 can, for example, be moved in a plane arranged parallel to the object plane 130. Further, the sample mount 126 can be move relative to the x-ray source 112 and relative to an x-ray detector assembly 134. In the orientation of FIG. 1, the sample mount 126 can be moved laterally in the x-direction and the y-direction.

The sample mount 126 including its support surface 128 has, for example, an opening 135 for transmission of x-rays 114 from the x-ray source 112 and/or for accommodating a protruding portion (not shown) of the x-ray source 112 at least partially. Instead of the opening 135, the sample mount 126 may also have an x-ray transmissive window (not shown) for transmission of x-rays 114. Furthermore, also the base 124 of the sample mount assembly 122 has an opening 135′ or x-ray transmissive window (not shown) similar as the sample mount 126.

The x-ray imaging system 100 further comprises a position-sensitive x-ray detector assembly 134 for detecting x-rays 114′ transmitted through the region of interest 104 of the sample 102. The position-sensitive x-ray detector assembly 134 comprises, for example, a scintillator element 136 at an entrance window of the x-ray detector assembly 134. The scintillator element 136 is configured to convert the incoming x-rays 114′ into light of longer wavelength (detectable light), e.g., ultraviolet light, visible light and/or infrared light. The x-ray detector assembly 134 further includes, for example, a detector unit 138 (e.g., a detector array, a CCD or CMOS array) for detecting the detectable light.

In the shown figures and the corresponding description, the x-ray detector assembly 100 includes a scintillator element 136 for converting incoming x-rays 114′ into detectable light and a detector unit 138 for detecting the detectable light. However, although not shown, in other examples, any herein described x-ray detector assembly may include instead of a scintillator element for converting incoming x-rays into detectable light and a detector unit for detecting the detectable light-also a detector unit for directly detecting x-ray light (and no scintillator element). In this case, the described features (e.g., of a flat geometric shape etc.) apply in an analog manner to the detector unit for directly detecting x-rays.

Although not shown in FIG. 1, the x-ray detector assembly 134 may also include a (magnifying or non-magnifying) optics unit for transferring the detectable light from the scintillator element 136 to the detector unit 138. Alternatively, the x-ray detector assembly 134 may include a (magnifying or non-magnifying) fiber optic plate for guiding the detectable light from the scintillator element 136 to the detector unit 138.

The x-ray imaging system 100 provides microscopic imaging. A magnification and, hence, a spatial resolution, of the x-ray imaging system 100 depends on the geometric magnification of the system 100. The geometric magnification of the system 100 is given by a ratio of a first distance between the x-ray source 112 and the x-ray detector assembly 134 and a second distance between the x-ray source 112 and the region of interest 104 of the sample 102.

The x-ray imaging system 100 comprises, for example, a control device 139 for reconstructing the three-dimensional images 108 based on the two-dimensional images 106.

The x-ray imaging system 100 is configured for x-ray imaging of a relatively large region of interest 104 which forms a substantial fraction of the entire sample 102.

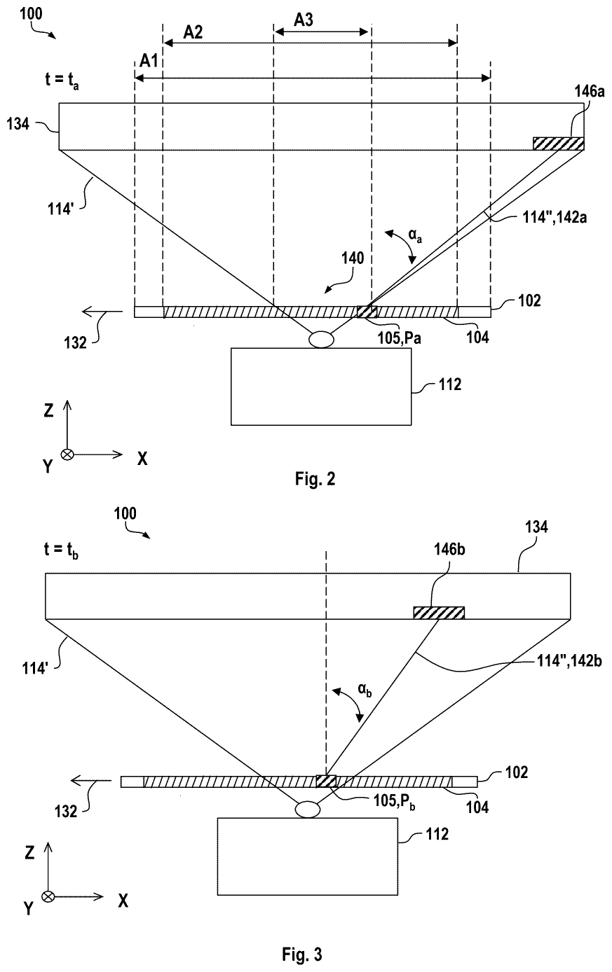

As indicated in FIG. 2, the sample 102 has, for example, a size A1 in the object plane 130 (first size A1) which is at least 1.000 mm², at least 3.000 mm², at least 5.000 mm², at least 10.000 mm², at least 30.000 mm², at least 50.000 mm² and/or at least 70.000 mm². Furthermore, a size A2 of the region of interest 104 in the object plane 130 (second size A2) is, for example, at least 0.01 times, at least 0.1 times, at least 0.3 times, at least 0.5 times, at least 0.7 times and/or at least 0.9 times of the size A1 of the sample 102. Although not visible in FIG. 2, both the first size A1 and the second size A2 are two-dimensional areas. In the orientation of FIG. 1, both the first size A1 and the second size A2 are areas in the xy-plane.

In other words, with the x-ray imaging system 100 three-dimensional x-ray images 106 of at least 10%, at least 30%, at least 50%, at least 70%, at least 90% and/or 100% of the entire sample 102 can be obtained.

In the example of FIGS. 1 to 5, the size A2 of the region of interest 104 is about 0.9 times (90%) of the size A1 of the sample 102.

For example, the x-ray detector assembly 134 is configured with a relatively large field-of-view 140. See FIG. 2 for a size A3 of the field-of-view 140. Note that also the third size A3 is a two-dimensional area. In addition, the size A2 of the region of interest 104 is much larger than the size A3 of the field-of-view 140.

For obtaining two-dimensional images 106 of the region of interest 104 including information for different projection angels α, the sample 102 is moved laterally within the object plane 130 and relative to the x-ray source 112. By moving the sample 102 laterally with respect to the x-ray source 112, each portion 105 of the region of interest 104 is viewed by the x-ray detector assembly 134 under the different projection angels α. In other words, by moving the sample 102 laterally with respect to the x-ray source 112, an angle of incident β of an x-ray beam 114″ hitting a specific exemplary portion 105 of the region of interest 104 is varied.

FIG. 1 shows the sample 102 with the exemplary portion 105 of the region of interest 104 in a lateral position P. The x-ray beam 114″ transmitted through the exemplary portion 105 of the region of interest 104 has a central axis 142. The angle of incident β is the angle by which the central axis 142 deviates from a surface normal 144, wherein the surface normal 144 is arranged perpendicular to the surface 128 of the sample mount 126 (i.e. perpendicular to the object plane 130). Note that α=β.

The central axis 142 can also be called an x-ray propagation axis for the exemplary portion 105 of the region of interest 104. The x-ray propagation axis 142 is extending from the x-ray source 112 (i.e., the source region 116 of the x-ray source 112), through the exemplary portion 105 of the region of interest 104 of the sample 102, and to the x-ray detector assembly 134.

FIGS. 2 to 5 illustrate a process of obtaining two-dimensional images 106 of the region of interest 104 for different projection angels α (i.e. different angles of incident β FIG. 1) for the exemplary portion 105 by moving the sample 102 laterally (arrow 132). For example, FIG. 2 illustrates the system 100 at a first point in time tₐ and shows the sample 102 with the exemplary portion 105 of the region of interest 104 at a first lateral position Pₐ. By taking an x-ray image 106 of the sample 102 with the exemplary portion 105 at the lateral position Pₐ, the exemplary portion 105 of the region of interest 104 is imaged onto the detector assembly 134 in the detector region 146a. FIG. 2 further illustrates a corresponding projection angle αₐ under which the exemplary portion 105 of the region of interest 104 is viewed by the detector assembly 134 in the lateral position Pₐ (x-ray propagation axis 142a).

The sample 102 is moved from the first lateral position Pₐ in FIG. 2 to a second lateral position P_{b} in FIG. 3. In the second lateral position P_{b} and at a second point in time t_{b}, the exemplary portion 105 of the region of interest 104 is imaged with a second projection angle α_{b} onto a second detector region 146b of the detector assembly 134 (second x-ray propagation axis 142b).

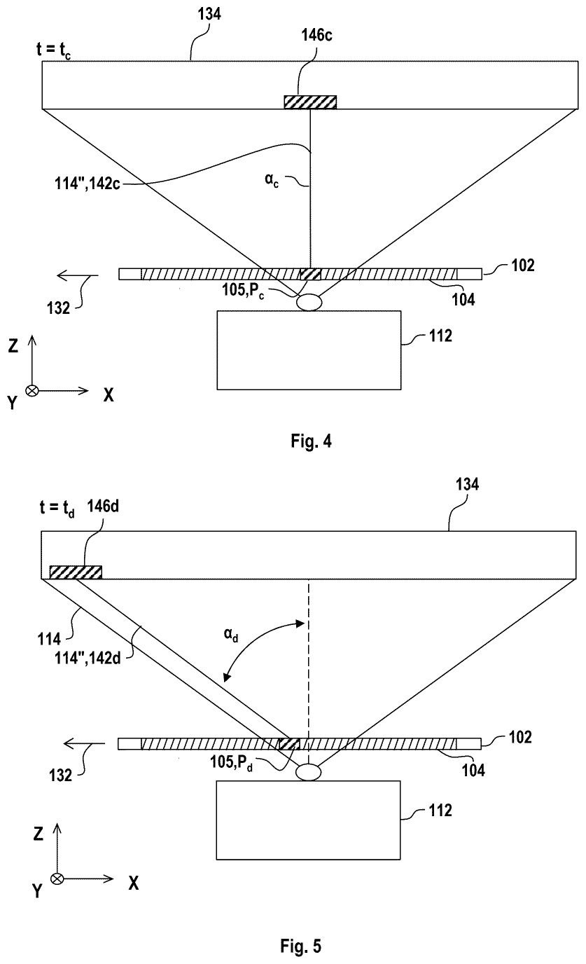

Then, the sample 102 is moved such that the exemplary portion 105 of the region of interest 104 is moved from the second lateral position P_{b} in FIG. 3 to a third lateral position P_{c} in FIG. 4. In the third lateral position P_{c} and at a third point in time t_{c}, the exemplary portion 105 of the region of interest 104 is imaged with a third projection angle α_{c} onto a third detector region 146c of the detector assembly 134 (third x-ray propagation axis 142c).

Likewise, the sample 102 is moved further such that the exemplary portion 105 of the region of interest 104 is moved from the third lateral position P_{c} in FIG. 4, to a fourth lateral position P_{d} in FIG. 5. In the fourth lateral position P_{d} and at a fourth point in time t_{d}, the exemplary portion 105 of the region of interest 104 is imaged with a fourth projection angle α_{d} onto a fourth detector region 146d of the detector assembly 134 (fourth x-ray propagation axis 142d).

FIGS. 1 to 5 show exemplary several lateral positions P, Pₐ to P_{d} of the exemplary portion 105 of the region of interest 104 of the sample 102. For reconstructing a three-dimensional x-ray image 108 of the region of interest 104, two-dimensional x-ray images 106 in many more than the shown lateral positions of the sample 102 are taken.

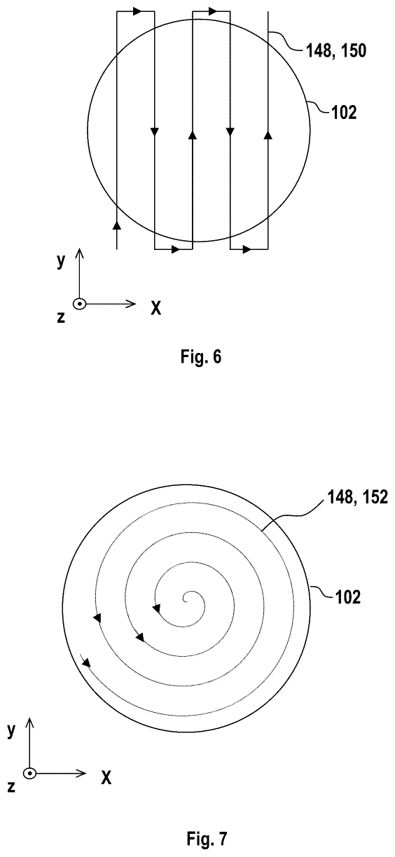

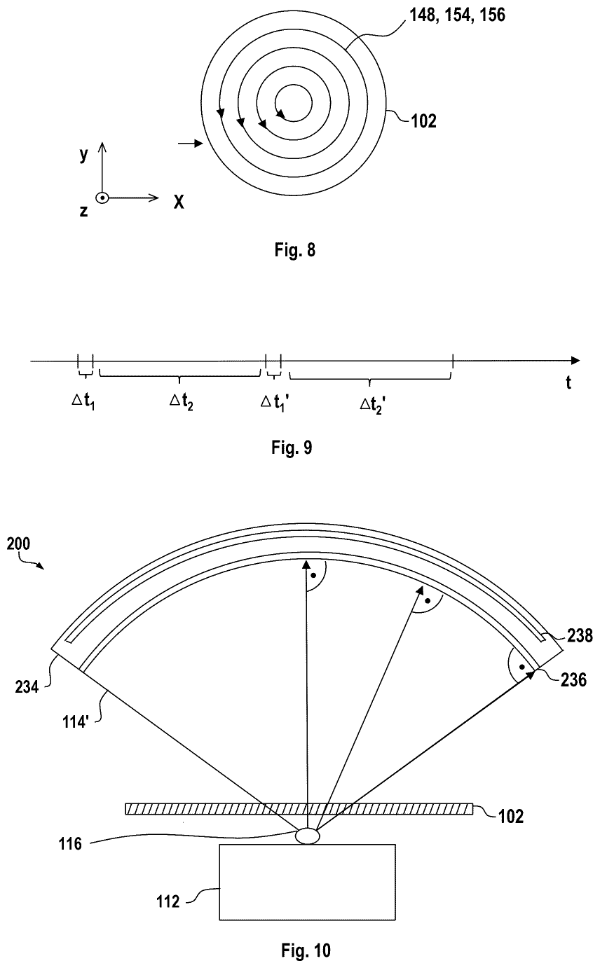

Furthermore, for obtaining the two-dimensional x-ray images 106 of the region of interest 104 used to reconstruct the three-dimensional x-ray image 108 of the region of interest 104, the sample 102 (e.g., the sample mount 126 together with sample 102) is moved laterally in a predetermined pattern 148. As shown in FIGS. 6 to 8, the predetermined pattern 148 may include a meander pattern 150 (FIG. 6), a spiral pattern 152 (FIG. 7) or a pattern 154 of concentric circles 156 (FIG. 8). Note that the size of the patterns 148 shown in FIGS. 6 to 8 are enlarged for illustration purposes, while moving the sample 102 may be carried out with a much higher spatial resolution than shown in these figures.

As illustrated in FIG. 9, the two-dimensional images 106 of the region of interest 104 for the different lateral positions P may be obtained-instead of by moving the sample 102 continuously-by a step-and-shoot process. In this approach, moving the sample 102 and exposing the sample 102 with x-rays 114 does not take place simultaneously but rather in two distinct time periods Δt₁, Δt₂. For example, the sample 102 is moved during a first time span Δt₁ quickly from a first lateral position (e.g., FIG. 2) to a second lateral position (e.g., FIG. 3) without obtaining x-ray images 106 of the region of interest 104 of the sample 102. Subsequently, the region of interest 104 of the sample 102 is irradiated during a second time span Δt₂ in the second lateral position (FIG. 3) with x-rays 114 without moving the sample 102. Then, the sample 102 is moved again from the second lateral position (FIG. 3) to a further lateral position in a further first time span Δt₁′ without carrying out an exposure. Subsequently the sample 102 is exposed to x-rays in a further second time span Δt₂′ without moving the sample 102 etc.

By using the step-and-shoot process for obtaining the x-ray images 106, a blur of the x-ray images 106 due to motion of the sample 102 during the x-ray exposure can be avoided.

In FIGS. 1 to 5, an x-ray detector assembly 134 is shown which has a detector unit 138 and a scintillator element 136 each having a flat shape 158, 160 and being arranged parallel to the object plane 130.

Having the x-ray detector assembly 134 with the detector unit 138 and the scintillator element 136 arranged parallel to the sample 102—instead of at an oblique angle—the x-ray detector assembly 134 can be arranged close to the sample 102 without the risk of colliding with the sample 102. In this configuration, the sample 102 is moved in a horizontal plane 130 between the x-ray source 112 and the x-ray detector assembly 134 instead of rotating the sample 102 around a vertical axis. As shown in FIGS. 2 to 5, the sample 102 is moved laterally such that the region of interest 104 of the sample 102 is moved through the x-ray cone 120 of the x-ray source 112. Arranging the x-ray detector assembly 134 close to the sample 102 allows x-ray imaging with a moderate geometric magnification. This can be desirable in case that a relatively large region of interest 104 should be imaged within a moderate time duration. The configuration shown in FIGS. 1 to 5 can result in a homogeneous geometric magnification over the pixels of the x-ray detector assembly 134. A blurring effect can be present because of an increasing projection angle α (e.g., α_{d} in FIG. 5) which leads to an increasing number of pixels of the detector unit 138 detecting a single x-ray beam 114″. In other words, a single x-ray beam 114″ is smeared out over an increasing number of pixels of the detector unit 138. This blurring effect occurs for every exemplary portion 105 within the region of interest 104, whenever this portion 105 is projected to an edge of the detector unit 138 of the x-ray detector assembly 134, i.e. for the cases shown in FIGS. 2 and 5.

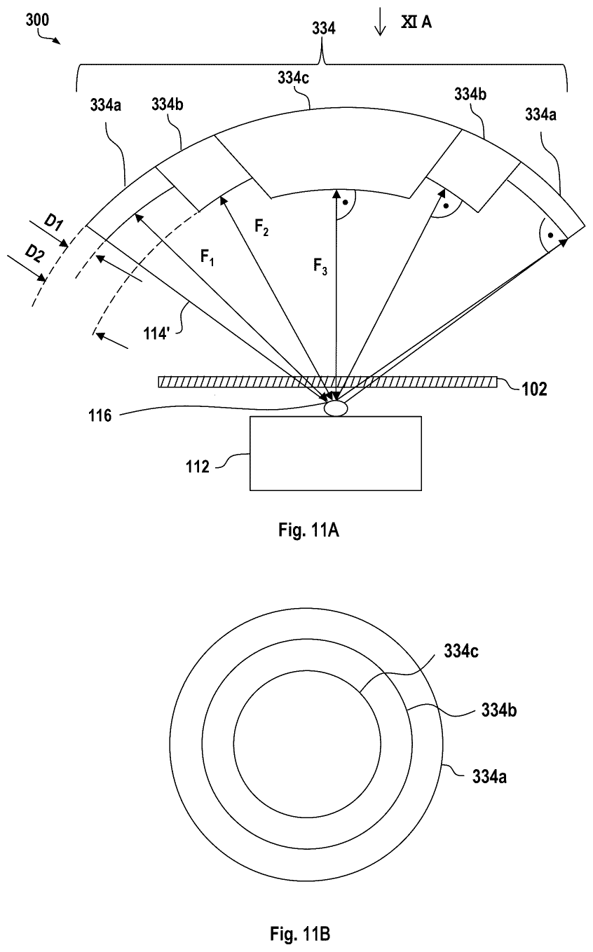

In FIG. 10, an x-ray imaging system 200 according to a further embodiment is shown. In the following, mainly only differences to the embodiment of FIG. 1 are described.

The x-ray imaging system 200 comprises an x-ray detector assembly 234. The x-ray detector assembly 234 comprises a detector unit 238 and a scintillator element 236 similar as the x-ray detector assembly 134 in FIG. 1. In contrast to the x-ray detector assembly 134 in FIG. 1, the x-ray detector assembly 234 has a curved and/or semi-spherical shape. For example, both the detector unit 238 and the scintillator element 236 of the x-ray detector assembly 234 have a curved and/or semi-spherical shape such that the x-ray source 112 faces a concave surface of the detector unit 238 and of the scintillator element 236.

In the example of FIG. 10, the detector unit 238 and the scintillator element 236 have, for example, each a semi-spherical shape with a source region 116 of the x-ray source 112 arranged in the center of a sphere corresponding to the semi-spherical shape. In this example, the scintillator element 236 and the detector unit 238 are arranged concentrically to each other.

Having a curved and/or semi-spherical shape of the detector unit 238 and the scintillator element 236, the x-rays 114, 114′ emitted from the source region 116 of the x-ray source 112 and transmitted through the region of interest 104 of the sample 102 impact a surface of the scintillator element 236 at normal incidence. Hence, a loss of spatial resolution due to blurring caused by an oblique passing of the x-rays 114′ through the scintillator element 236 is avoided.

The geometric magnification of the system 200 may no longer be constant across the field-of-view 140 of the detector assembly 234, because a distance (second distance) between the x-ray source 112 and the sample 102 is not constant for each pixel of the detector unit 238 due to the flatness of the sample 102. Since the geometric magnification depends on this distance, the geometric magnification also varies across the field-of-view 140 of the x-ray detector assembly 234.

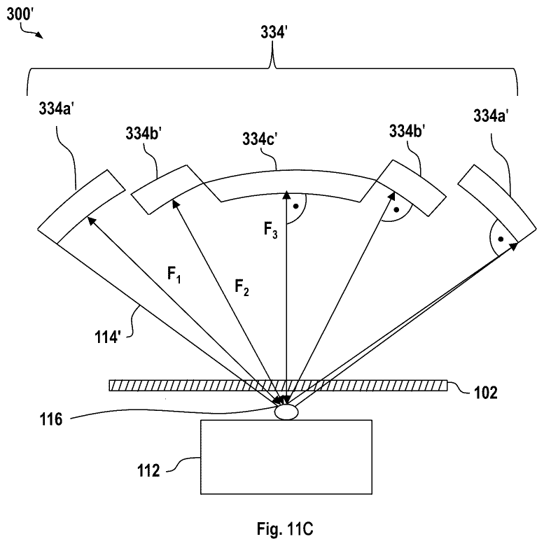

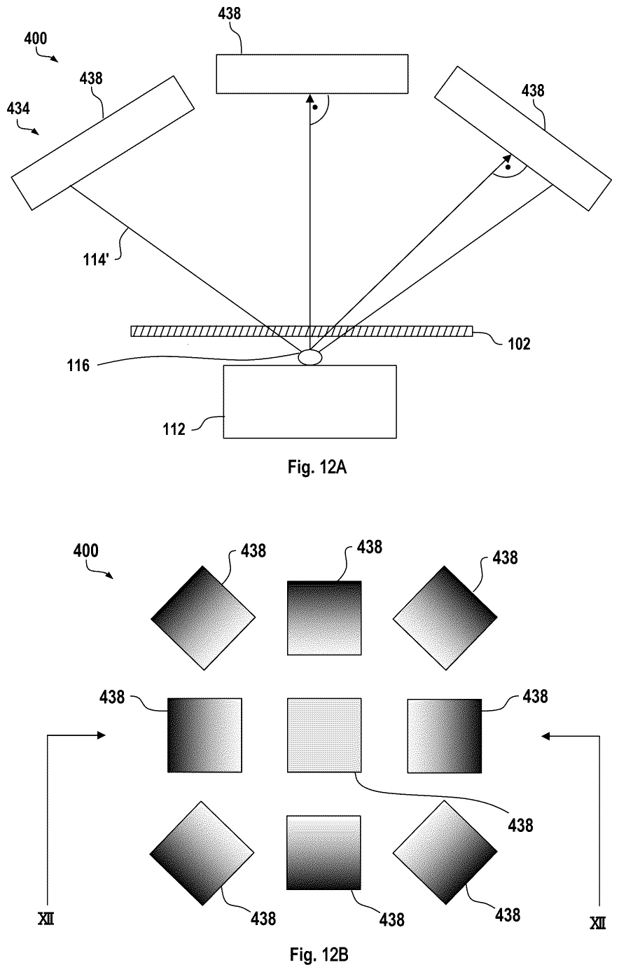

In FIG. 11A, an x-ray imaging system 300 according to a further embodiment is shown. In the following, mainly only differences to the embodiment of FIG. 1 are described.

The x-ray imaging system 300 comprises an x-ray detector assembly 334. In contrast to the x-ray detector assembly 134 in FIG. 1, the x-ray detector assembly 334 comprises multiple detector segments 334a to 334c. Further, each segment 334a to 334c comprises a detector unit and a scintillator element similar as the x-ray detector assembly 134, 234 in FIGS. 1, 10. Furthermore, each segment 334a to 334c has a curved and/or semi-spherical shape. In the example of FIG. 11A, each detector segment 334a to 334c is a rotationally symmetric section of a hemisphere. In FIG. 11B, a top view of the segments 334a to 334c is shown (as viewed along the arrow XIA in FIG. 11A). In the example of FIG. 11A, the segments 334a to 334c have different thicknesses D1, D2. Alternatively to having different thicknesses D1, D2, some or all of the detector segments 334a′ to 334c′ may also have the same and/or similar thicknesses D1, D2 and may be shifted in the radial direction with respect to each other, as shown in FIG. 1C. In both cases (FIG. 11A and FIG. 11C), a distance F1-F3 between the source region 116 of the x-ray source 112 and a surface of a corresponding segment 334a-334c or 334a′-334c′ facing the source region 116 varies such that a distance difference between the source region 116 of the x-ray source 112 and the sample 102 for different detector pixels of the detector units of the x-ray detector assembly 334, 334′ is compensated. Hence, a more homogeneous magnification for the pixels of the x-ray detector assembly 334, 334′ is achieved compared to the case shown in FIG. 10. The detector segments 334a to 334c and 334a′ to 334c′ of the x-ray detector assembly 334, 334′ can be described as Fresnel-like segments (in analogy to a Fresnel lens).

In FIG. 12A, an x-ray imaging system 400 according to a further embodiment is shown. In the following, mainly only differences to the embodiment of FIG. 1 are described. The x-ray imaging system 400 comprises an x-ray detector assembly 434. The x-ray detector assembly 434 comprises multiple detector segments 438. The detector segments 438 are appropriately tilted with respect to each other to approximate and/or conform to a curved and/or semi-spherical shape. Further, each detector segment 438 comprises a (e.g., flat) scintillator element, a (e.g., flat) transfer unit and a (e.g., flat) detector unit similar as the x-ray detector assembly 134 in FIG. 1.

FIG. 12B shows a top view of a portion of the x-ray imaging system 400 of FIG. 12A. For example, with respect to the multiple detector segments 438, FIG. 12A can be seen as cross-section along line XII-XII in FIG. 12B. Exemplarily, the x-ray imaging system 400 shown in FIG. 12B comprises nine detector segments 438 inclined relative to each other. A degree of inclination of each detector segment 438 is indicated schematically in FIG. 12B.

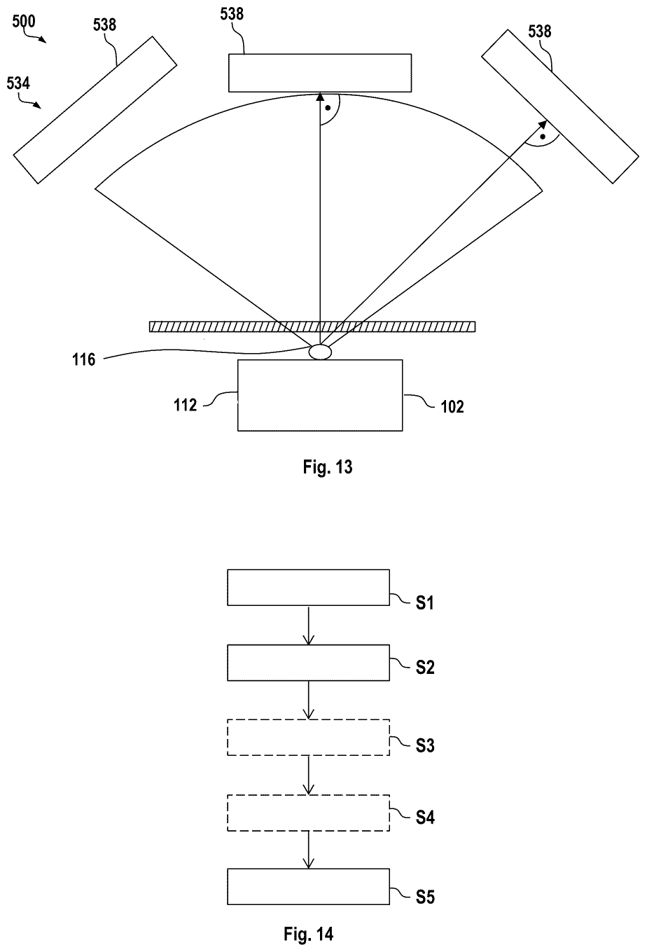

In FIG. 13, an x-ray imaging system 500 according to a further embodiment is shown. In the following, mainly only differences to the embodiment of FIG. 1 are described. The x-ray imaging system 500 comprises an x-ray detector assembly 534. The x-ray detector assembly 534 comprises multiple detector segments 538. The detector segments 538 are appropriately tilted with respect to each other. Further, each detector segment 538 comprises a (e.g., flat) scintillator element, a (e.g., flat) transfer unit and a (e.g., flat) detector unit similar as the x-ray detector assembly 134 in FIG. 1. In addition, the tilted detector segments 538 are arranged at different distances from the source region 116 of the x-ray source 112 representing the Fresnel-like concept of FIG. 11A.

In other examples, the x-ray imaging system 400 and/or 500 (FIGS. 12, 13) may comprise one or more curved and/or semi-spherical scintillator elements (not shown), one or more fiber optic plates (not shown) and multiple flat detector units arranged tilted to each other. The multiple flat detector units are, for example, arranged similar as the detector segments 438 in FIG. 12A or similar as the detector segments in FIG. 13. In this example, the fiber optic plate(s) is configured for guiding the detectable light from the curved scintillator element to the flat detector units.

In the following, a method for obtaining three-dimensional x-ray images 108 of the sample 102 is described with reference to FIG. 14.

The method is described exemplary for the x-ray imaging system 100 shown in FIG. 1. However, the method can also be carried out by using any of the herein other described x-ray imaging systems 200 to 500 (FIGS. 10 to 13).

In a first step S1 of the method, a sample 102 is arranged in an object plane 130 of an x-ray imaging system 100. The sample 102 has, for example, a first size A1 in the object plane 130 of at least 1.000 mm². However, the first size A1 may also have another value.

In a second step S2 of the method, two-dimensional x-ray images 106 of a region of interest 104 of the sample 102 are obtained including information for different projection angles α. The region of interest 104 has a second size A2 in the object plane of at least 0.01 times the first size A1.

During step S2, the sample 102 is moved laterally as described above with reference to FIGS. 1 to 9.

The two-dimensional x-ray images 106 of the region of interest 104 used to reconstruct the three-dimensional x-ray image 108 of the region of interest are, for example, obtained within a relatively short time span of only a few hours or less than one hour.

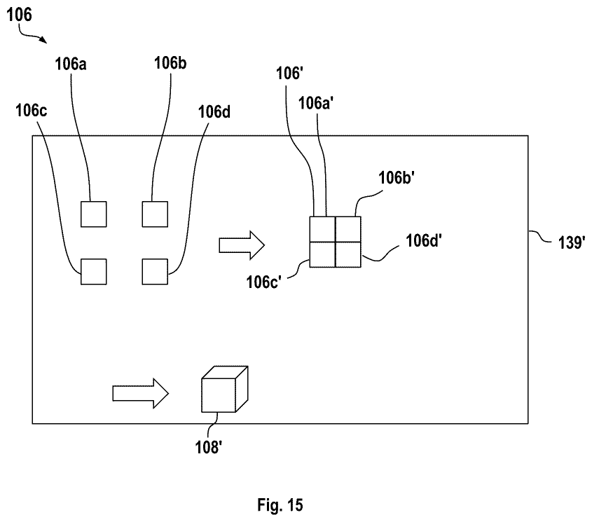

In embodiments in which the x-ray detector assembly 334, 334′, 434, 534 (FIGS. 11 to 13) comprises multiple segments (e.g., segments 334a-334c in FIG. 11A or segments 438 in FIG. 12A etc.), each obtained two-dimensional image 106 of the region of interest 104 is a composite of several x-ray sub-images 106a-106d corresponding to the different detector segments. This is illustrated in FIG. 15 which shows a control device 139′ of the x-ray imaging system 100 according to a further embodiment. Exemplarily, one obtained x-ray image 106 is shown including four sub-images 106a-106d. Before combining the sub-images 106a-106d to a combined image 106, the individual sub-images 106a-106d may be corrected computer-implemented, for example based on a geometric shape, tilting angle and/or position of the corresponding detector segment (e.g., segments 334a-334c in FIG. 11A or segments 438 in FIG. 12B etc.). The results are corrected two-dimensional sub-images 106a′-106d′. Then, a corrected two-dimensional x-ray image 106′ is synthesized based on the corrected sub-images 106a′-106d′.

In a third step S3 of the method, a three-dimensional x-ray image 108 is reconstructed computer-implemented based on the obtained two-dimensional x-ray images 106.

In case, that in step S2, corrected images 106′ are determined, the three-dimensional x-ray image 108′ is reconstructed in step S3 based on the corrected two-dimensional x-ray images 106′.

Although the present disclosure has been described in accordance with certain embodiments, it is obvious for the person skilled in the art that modifications are possible in all embodiments.

### REFERENCE NUMERALS

100 System
102 Sample
104 Region of interest
106, 106′ Image
106a-106d Image
106a′-106d′ Image
108, 108′ Image
110 Wafer
112 Source
114 X-ray
114′, 114′′ X-ray
116 Source region
118 Beam
120 Cone
122 Sample mount assembly
124 Base
128 Surface
130 Object plane
132 Direction
134 X-ray detector assembly
135, 135′ Opening
136 Scintillator element
138 Detector unit
139, 139′ Control device
140 Field-of-view
142 Axis
142a-142d Axis
144 Surface normal
146a-146d Detector region
148 Pattern
150 Pattern
152 Pattern
154 Pattern
156 Circle
158 Shape
160 Shape
200 X-ray imaging system
234 X-ray detector assembly
236 Scintillator element
238 Detector unit
300, 300′ X-ray imaging system
334, 334′ X-ray detector assembly
334a-334c Segment
334a′-334c′ Segment
400 X-ray imaging system
434 X-ray detector assembly
436 Scintillator element
438 Segment
500 X-ray imaging system
534 X-ray detector assembly
536 Scintillator element
538 Segment
α Angle
αₐ-αd Angle
β Angle
Δt₁, Δt₁′ time span
Δt₂, Δt₂′ time span
A1, A2, A3 Size (area)
D1, D2 Thickness
E1 Plane
F1-F3 Distance
P Position
Pₐ-Pd Position
S1-S5 Method steps
t, tₐ-td time
x, y, z Direction

What is claimed is:

1. A method, comprising:
a) arranging a sample in an object plane of an x-ray imaging system, the sample having a first size in the object plane;
b) obtaining two-dimensional x-ray images of a region of interest of the sample comprising comprising information for different projection angles, the region of interest having a second size in the object plane, the second size being at least 0.01 times the first size; and
c) reconstructing, via a computer-implemented process, a three-dimensional x-ray image of the region of interest based on the two-dimensional x-ray images.

2. The method of claim 1, comprising obtaining the two-dimensional x-ray images of the region of interest by an x-ray detector assembly having a field-of-view with a third size, wherein the second size is greater than the third size.

3. The method of claim 2, wherein the second size is at least 10 times greater than the third size.

4. The method of claim 1, comprising obtaining the two-dimensional x-ray images of the region of interest within a time span of four hours or less.

5. The method of claim 1, further comprising:
obtaining the two-dimensional x-ray images using an x-ray source irradiating the region of interest with x-rays and an x-ray detector assembly detecting x-rays transmitted through the region of interest; and
during b), laterally moving the sample during within the image plane and relative to the x-ray source and the x-ray detector assembly so that each portion of the region of interest is irradiated by x-rays of an x-ray cone of the x-ray source under different angles of incident for the different lateral positions to obtain the two-dimensional x-ray images of the region of interest including the information for the different projection angles.

6. The method of claim 5, comprising moving the sample laterally in at least one pattern selected from the group consisting of a meander pattern, a spiral pattern, and a pattern of concentric circles.

7. The method of claim 5, comprising obtaining the two-dimensional images of the region of interest by a step-and-shoot process, wherein the step-and-shoot process comprises, for each of multiple predetermined lateral positions of the sample:
laterally moving the sample to the predetermined lateral position without obtaining x-ray images of the region of interest during a first time span; and
irradiating the region of interest in the predetermined lateral position with x-rays without moving the sample during a second time span.

8. The method of claim 1, comprising obtaining the two-dimensional x-ray images by irradiating the region of interest so that each angle of incidence of the x-rays hitting the region of interest is 60° or less.

9. One or more machine-readable hardware storage devices comprising instructions that are executable by one or more processing device to perform operations comprising the method of claim 1.

10. A system comprising:
one or more processing devices; and
one or more machine-readable hardware storage devices comprising instructions that are executable by one or more processing device to perform operations comprising the method of claim 1.

11. A method, comprising:
obtaining two-dimensional x-ray images of a region of interest of a sample in an object plane of an x-ray imaging system, the two-dimensional x-ray images comprising information for different projection angles, the sample having a first size in the object plane, the region of interest having a second size in the object plane, the second size being at least 0.01 times the first size; and
reconstructing, via a computer-implemented process, a three-dimensional x-ray image of the region of interest based on the two-dimensional x-ray images.

12. A system comprising:
one or more processing devices; and
one or more machine-readable hardware storage devices comprising instructions that are executable by one or more processing device to perform operations comprising the method of claim 11.

13. A system comprising:
one or more processing devices; and
one or more machine-readable hardware storage devices comprising instructions that are executable by one or more processing device to perform operations comprising the method of claim 11.

14. An x-ray imaging system, comprising:
a moveable sample stage configured to support a sample in an object plane of the x-ray imaging system;
an x-ray source configured to emit toward a region of interest of the sample; and
an x-ray detector assembly configured to detect x-rays transmitted through the region of interest of the sample,
a controller configured to control components of the x-ray imaging system to:
obtain two-dimensional x-ray images of the region of interest of the sample, the two-dimensional images comprising information for different projection angles, the sample having a first size in the object plane, the region of interest having a second size in the object plane, and the second size being at least 0.01 times the first size; and
reconstruct, via a computer implemented process, a three-dimensional x-ray image of the region of interest based on the two-dimensional x-ray images.

15. The x-ray imaging system of claim 14, wherein the x-ray detector assembly has a field-of-view with a third size, and the second size is greater than the third size.

16. The x-ray imaging system of claim 14, comprising a sample stage assembly which comprises the sample stage, wherein the sample stage assembly is configured to move the sample laterally, parallel to the image plane and relative to the x-ray source and the x-ray detector assembly.

17. The x-ray imaging system of claim 16, wherein the x-ray imaging system is configured to move the sample laterally relative to the x-ray source to different predetermined lateral positions so that each portion of the region of interest is irradiatable by x-rays of an x-ray cone of the x-ray source under different angles of incidence for the different lateral positions to obtain the two-dimensional x-ray images of the region of interest comprising information for the different projection angles.

18. The x-ray imaging system of claim 14, wherein the x-ray detector assembly comprises at least one member selected from the group consisting of a detector unit and a scintillator element, the at least one member having a flat shape and being arranged parallel to the object plane.

19. The x-ray imaging system of claim 14, wherein the x-ray detector assembly comprises at least one member selected from the group consisting of a detector unit and a scintillator element, the at least one member having a curved and/or semi-spherical shape so that the x-ray source faces a concave surface of the at least one member.

20. The x-ray imaging system of claim 14, wherein the x-ray detector assembly comprises:
a first plurality of segments, each segment of the first plurality of segments comprising at least one member selected from the group consisting of a flat detector unit and a flat scintillator element, the at least one member being parallel to the object plane;
a second plurality of segments, each segment of the second plurality of segments having a curved and/or semi-spherical shape;
a plurality of Fresnel-like segments; and/or
a third plurality of segments tilted relative to each other to approximate a curved and/or semi-spherical shape.
